(19) United States
(12) Patent Application Publication
(10) Pub. No.: US 2014/0135592 A1
(43) Pub. Date: May 15, 2014

(54) HEALTH BAND

(71) Applicant: dacadoo ag, Zurich (CH)

(72) Inventors: Peter Ohnemus, Kusnacht (CH); Jesper Ohnemus, Odense M. (DK); Andre Naef, Zurich (CH); David Leason, Chappaqua, NY (US)

(73) Assignee: dacadoo ag, Zurich (CH)

(21) Appl. No.: 14/079,495

(22) Filed: Nov. 13, 2013

Related U.S. Application Data
(60) Provisional application No. 61/725,924, filed on Nov. 13, 2012.

(51) Int. Cl. A61B 5/00; A61B 5/0205; A61B 5/145; A61B 5/11; A61B 5/01

(52) U.S. Cl. CPC A61B 5/681; A61B 5/1118; A61B 5/0022; A61B 5/742; A61B 5/7495; A61B 5/4806; A61B 5/01; A61B 5/14532; A61B 5/4266; A61B 5/02055; A61B 5/4866; A61B 5/1112; A61B 5/021
USPC 600/301; 600/300; 600/595; 600/549; 600/485; 600/365; 600/307; 600/508; 600/483; 600/547

(57) ABSTRACT

A wearable device is disclosed configured to provide and transmit user information. Health-related information that is associated with a user information is received and output to a user. The wearable device can include a display configured to provide a user interface and a sensor senses information associated with biological features, physiological features and/or physical activity of the user, while the device is being worn. A communications subsystem is provided that is configured to communicate with the remote computing device. The wearable device includes a processing subsystem is provided and configured to process the sensed information to provide processed user information. The processed user information is transmitted via the communication subsystem, to the remote computing device, and health-related information associated with the processed user information is received from the remote computing device. The health-related information is provided, via the user interface, on the display.

### CROSS REFERENCE TO RELATED APPLICATIONS

The present application claims priority to U.S. patent application Ser. No. 61/725,914, filed Nov. 13, 2012, the contents of which are hereby incorporated by reference in its entirety.

### FIELD

The present application relates, generally, to information communication systems, and, more particularly, to a networked system and method for displaying information.

### BACKGROUND

Advances in providing an individual with access to information regarding his or her health have been made, including for conveying health-related information remotely. Advances have further been made to protect a person from accidental disclosure of sensitive information by masking health-related information in a calculated numerical value.

One such advancement is described in commonly assigned PCT Patent Application PCT/US11/53971, in which a unique health score computation method is disclosed which masks underlying health statistics, yet provides a benchmark for a variety of applications. Health-related information is collected and processed into a composite numerical value. The value may, thereafter, be published without fear of divulging a person's private health-related information.

Examples of information that is collected and analyzed for calculating a health score include a plurality of intrinsic and extrinsic parameters of a user. Weighting factors may also be applied to the parameter(s) in order control the relative affect that each parameter has on the user's calculated numerical health score. The health score may be computed by combining the weighted parameters in accordance with an algorithm, and the value may be published via a portal, while the underlying parameters remain private. In one implementation, the portal is an internet based information sharing forum.

### SUMMARY

In one or more implementations, a wearable device is provided that is configured to provide user information, and to transmit the user information to a remote computing device. Health-related information that is associated with the user information is received and useable for outputting, to a user. In one or more implementations, the wearable device includes a display that is configured to provide a user interface. Further at least one sensor that is respectively configured to sense information while the device is being worn is provided, wherein the sensed information is of the type that is associated with biological features, physiological features and/or physical activity of the user. Further, a communications subsystem is provided that is configured to communicate with the remote computing device. The wearable device includes a processing subsystem is provided that includes a processor and processor readable media, the processing subsystem configured to process the sensed information to provide processed user information. The processed user information is transmitted via the communication subsystem, to the remote computing device, and health-related information associated with the processed user information is received from the remote computing device. The health-related information is provided, via the user interface, on the display.

In one or more implementations, the health-related information is formatted as a health score that is calculated by applying a decay component to reduce a relative weight of the sensed information associated with the physical activity of the user. The processed user data is further transformed into a masked composite numerical value. The health score is provided via the user interface on the display free of human intervention.

The details of one or more implementations are set forth in the accompanying drawings and the description below. Other features and advantages will be apparent from the description and drawings, and from the claims.

### BRIEF DESCRIPTION OF THE DRAWINGS

FIG. 1 shows an example hardware arrangement for obtaining, processing, and outputting content in accordance with an implementation;

FIG. 2 is a block diagram that illustrates functional elements of a computing device in accordance with an embodiment;

FIGS. 3-6 illustrate various implementations of a health hand 101 in accordance with the present application;

FIG. 7 is a schematic flow diagram according to one embodiment of the present application;

FIGS. 8-12 are example screen shots of a user interface according to one embodiment of the present application;

FIG. 13 is an illustration of progressions over time of parameters used to determine the health score in one embodiment of the invention;

FIG. 14A is an illustration of a data presentation format according to one embodiment of the invention;

FIG. 14B is an illustration of a data presentation format according to one embodiment of the invention;

FIG. 14C is an illustration of a data presentation format according to one embodiment of the invention; and

FIG. 15 illustrates a data presentation format associated with plurality of user scores.

### DETAILED DESCRIPTION

The present application includes a wearable band that is configured to, among other things, obtain, process, send, receive and display various kinds of information, such as associated with a wearer's current personal health score or other health-related information. As used herein, the wearable band is generally referred to as a “health band” at least in part, for its ability to provide health score information to the wearer. Although many of the examples provided herein relate to a wearable circular-shaped band, other implementations are provided that support other shapes and sizes. For example, the device may be configured in rectangular, square, kidney, oblong or triangle shapes, and may be worn in various ways, such as pinned, strapped, buttoned, hung and clipped. Further, health-related information and health scores, various other kinds of information can be sent, received and/or displayed by the wearable band in accordance with the present application.

In one or more implementations, the device in accordance with the present application may be configured to include a head-worn display that is configured to send, receive and display information as shown and described herein. For example, the present application may be configured with or in GOOGLE GLASS.

In one or more implementations, the wearable band of the present application includes control modules that cooperate to provide the features described herein. As used herein, the term, “module,” refers, generally, to one or more discrete components that contribute to the effectiveness of the systems and methods described herein. Modules can include software elements, including but not limited to functions, algorithms, classes and the like. Modules also include hardware elements, substantially including those described below. Modules can operate independently or, alternatively, may depend upon one or more other modules in order to function. Each of the modules may include hardware, firmware, and/or software code executing in a processor, or some combination thereof, that configures the health band to implement at least some of the functionality described herein. The functionality of these modules can be combined or further separated, as understood by persons of ordinary skill in the art, in analogous implementations the present application.

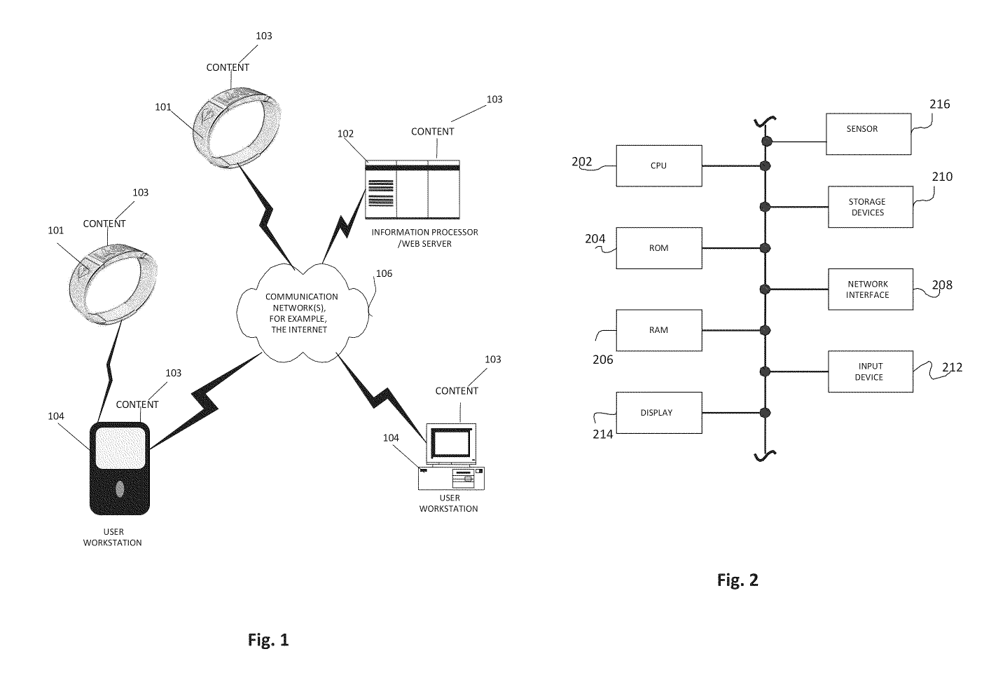

Referring now to the drawing figures, in which like reference numerals refer to like elements, FIG. 1 shows an example hardware arrangement for obtaining, processing, and outputting content, including via a communication network, such as the Internet. As shown in FIG. 1, an information processor 102, optionally functioning, as an internet web server, may send and receive electronic content 103 to/from a health band 101. Content 103 may include, for example, numerical, textual, graphical, pictorial, audio and video material. Such communications may occur between the information processor 102 and the health band 101 via a user workstation 104, which may be a mobile computing device such as a smart phone, tablet computer or other device, such as a laptop computer or desktop computer. Alternatively, such communications may occur between the information processor 102 and the health band 101 without the use of user workstation 104. Thus, in one or more implementations, the health band 101 may employ hardware and software modules that collect and/or receive information, process information, and transmit information between the health band 101 and information processor 102 and/or between the health band 101 and the user workstation 104. Information processor 102 may employ software for enabling a communication session, for example an HTTP session and/or a secured HTTPS session, to be established between the information processor 102 and the user workstation 104 and/or the health band 101.

In one or more implementations, the health band 101 includes hardware and software modules that configure the health band 101 to communicate via BLUETOOTH with the user workstation 104. Alternatively, the health band 101 may communicate via other suitable protocols, such as ANT+, Bluetooth Low Energy (“BLE”) or ZigBee, and with another device. The user workstation 104 may communicate securely with services provided by information processor 102 or other device over communication network 106 or over a different network (such as a local area network). Accordingly, different communication architectures may be employed by the present application.

In addition to communications-related modules, the health band 101 may include hardware and/or software modules that configure the health band 101 to track various forms of biological/physiological characteristics, exercise and/or activity of the wearer. For example, the health band 101 may be configured with one or more sensors and software that collect information relating to biological/physiological characteristics of the wearer, such as temperature, heart rate, blood pressure, perspiration or the like. In addition, the health band 101 can be configured to detect and/or display humidity associated with user's skin surface. Humidity information is usable, for example, to detect that the user is or is getting dehydrated and should drink. Sensors can be used to directly collect health information about a user and report that information. The biosensor can be placed in contact with the user's body to measure vital signs or other health related information from the user. For example, the biosensor can be a puke meter that is worn by the user in contact with the user's body so that the pulse of the user can be sensed, a heart rate monitor, an electrocardiogram device, a pedometer, a blood glucose monitor or one of many other devices or systems The biosensor can include a communication module (e.g., communication subsystem) so that the biosensor can communicate, either wired or wirelessly, the sensed data. The biosensor can communicate the sensed data to the user interface device, which in turn communicates that information to the microcontroller. Optionally, the biosensor can directly communicate the sensed the data to the microprocessor. The use of biosensors provides a degree of reliability in the data reported because it eliminates user error associated with manually, self-reported data.

In one or more implementations, the sensors that are provided in accordance with the present patent application can be non-invasive or invasive. For example, the sensor(s) can detect heartbeats and can provide for transmitting data from an implanted pacemaker. Alternatively, blood sensors that are mounted in a person's body transmit data that are usable by the health band 101, for example, to detect one or more marker proteins that may be present in the wearer's blood. Thus, the present application is usable with one or more sensors that are placed in or with the wearer's body, and/or are otherwise configured to communicate with devices that are implanted in a person.

The health band 101 may further be configured with components and software to enable the health band 101 to operate as a pedometer and to track steps taken by the wearer. This may include, for example, information representing the kinds of steps taken by the wearer of the health band 101. Such information relating to the steps may be, for example, identified by interval, size and/or by intensity. A number of steps taken in quick succession, for example, may be more strenuous than taken over a long period of time. Moreover, uphill steps may be more strenuous than downhill steps. These and other factors may be included with the exercise/activity data from health band 101.

The health band 101 may be configured to display number of calories burned over a period of time, such as one day, based at least on information received from the health band 101 (e.g., when operating as a pedometer). Moreover, the health band 101 may be configured with global positioning system (“GPS”) technology, which may provide location information regarding altitude, steepness of steps, or other relevant geographic information.

In another example the health band 101 may be configured to track swimming activity of the wearer. In other examples, the health band 101 may be configured to determine various physiological information of the wearer, such as heart rate, temperature, blood pressure, or the like. The health band 101 may further be configured with a galvanic skin sensor, for example, to measure stress levels of the wearer. Moreover, the health band 101 may be configured for sleep monitoring of the wearer as well. In one or more implementations, the health band 101 is configured with memory for storing information associated the wearer. In one or more other implementations, DNA information and/or one or more biomarkers is accessible, for example, to examine biological processes, pathogenic processes, or pharmacologic responses, such as associated with one or more therapies.

In addition to obtaining and/or generating information substantially automatically, information regarding exercise and/or other activity may be input by the wearer, including via a user interface via BLUETOOTH or other suitable communications with the user workstation 104.

In addition to tracking and monitoring the wearer, the health band 101 may be configured to send and/or receive information to/from the information processor 102. For example, daily activity, such as relating to exercise, may be transmitted to the information processor 102. Information received by the information processor 102 from the health band 101 may then be used for calculating the wearer's health score, which may be transmitted from the information processor 102 and received by and displayed on the health band 101. The information processor 102 may receive information from the health band 101, and may use the information in the calculation of the health score, such as shown and described in commonly assigned PCT Patent Application PCT/US11/53971. Thus and in accordance with the present application, health band 101 may serve as a source of information (e.g., relating to steps taken by the wearer) for one or more other devices, and may further provide information to the wearer that is received from one or more other devices (e.g., to display the wearer's health score). Accordingly, the health band 101 may operate to send, receive, process and display information, including health-related information.

In addition to receiving and transmitting information associated with the wearer, the health band 101 may be configured to process information, such as in connection with information collected and/or processed by sensing, components of the health hand 101. “Raw” data, which may be in the form of signals, may be collected by the health band 101 and processed to represent biological and/or physiological information. The health band 101 may be further configured with one or more algorithms stored in a memory which, when executed by a processor, calculates information, such as the wearer's health score (e.g., as shown and described in commonly assigned PCT Patent Application PCT/US11/53971). The wearer's health score (and/or the processed biological/physiological information) may be, thereafter, transmitted by the health band 101 to the information processor 102 for further processing, such as for ranking the wearer's health score among a plurality of people. In addition or in the alternative, the health band 101 can be configured with program instructions that enable the health band 10 to calculate a wearer's health score. Additional information, such as related to the ranking, may then be transmitted to the health band 101 and used to be displayed thereon.

In an example configuration, the following may be provided in connection with the health band 101, information and processor 102 and/or user workstation 104. Information, such as relating to a health score, is received by the health band 101 from the information processor 102. The information may be received via BLUETOOTH from user workstation 104, or may be received in other suitable ways. In addition to the health score, various forms of information, such as body information and parameters such as weight and step length may be used to facilitate, for example, an on-device estimation of calories expended and/or distance traveled. Other information that may be received includes time-correlated workout information, which may prevent double-counting of steps (pedometer) and workouts, and may also include workouts in accordance with an estimation of calories. Such estimations may be calculated by the health band 101.

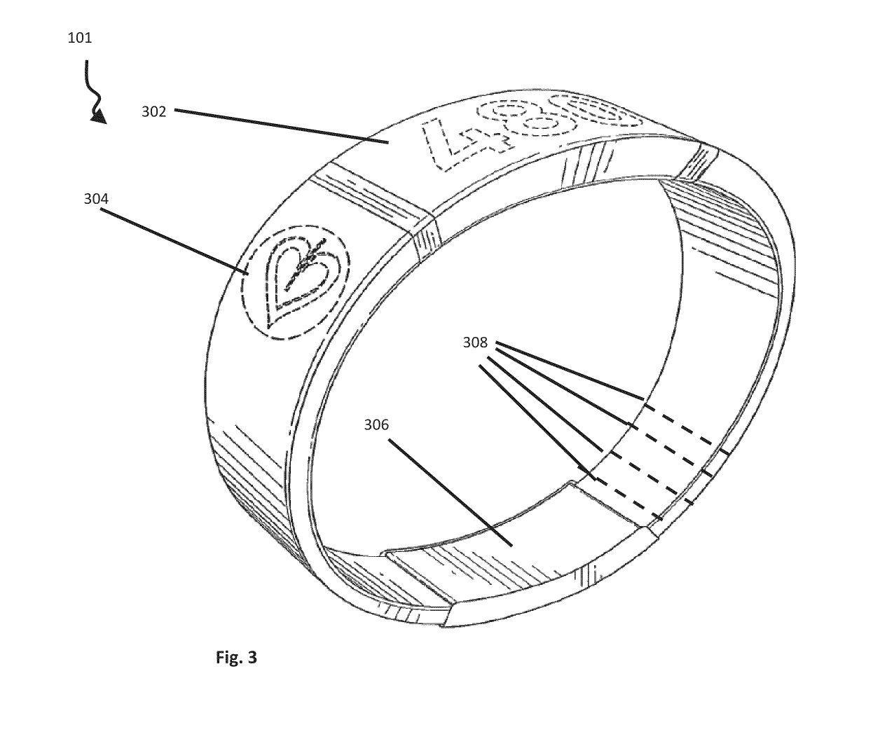

FIG. 2 illustrates one or more functional elements associated with health band 101, information processor 102, and/or user workstation 104, and including a processing subsystem that includes include one or more central processing units (CPU) 202 used to execute software code and control the operation of information processor 102 and/or health band 204, and processor-readable media including, for example, read-only memory (ROM) 204, random access memory (RAM) 206, as well as one or more network interfaces 208 to transmit and receive data to and from other computing devices across a communication network, storage 210 such as a solid state memory, a hard disk drive, CD ROM or DVD ROM for storing program code, databases and application data, one or more input devices 212 such as a keyboard, mouse, track ball, virtual keyboard, touchscreen, microphone and the like, and a display 214. As noted, the health band 101 may further be configured with one or more sensors 216 for sensing biological and/or physiological characteristics of the wearer.

It is contemplated herein that any suitable operating system can be used on the health band 10, the information processor 102 and/or the user workstation 104, for example, DOS, WINDOWS 3.x, WINDOWS 95, WINDOWS 98, WINDOWS NT, WINDOWS 2000, WINDOWS ME, WINDOWS CE, WINDOWS POCKET PC, WINDOWS XP, WINDOWS VISTA, MAC OS, UNIX, LINUX, PALM OS POCKET PC, IOS, DROID, BLACKBERRY, any other suitable operating system, for example in accordance with a respective device.

FIGS. 3-6 illustrate various implementations of a health band 101 in accordance with the present application.

In accordance with the example illustrated in FIG. 3, the health band 101 may include a display portion 302 configured to operate in a plurality of modes and display information corresponding to the modes. For example, information representing a wearer's health score may be displayed in one mode, a number of calories burned during a particular hike may be displayed in another mode, and the time of day and/or date may be displayed in yet another mode. In the example shown in FIG. 3, the value “480” is displayed that represents the wearer's current health score. The present application is useful to provide current, up-to-date information associated with a wearer's health (e.g., the wearer's health score), for example, as the wearer is engaging in physical activity. Thus, the health band 101 of the present application provides up-to-the-minute health score (and other) information to the wearer, thereby keeping the wearer motivated to work hard, stay focused on achieving goals, and be successful in health-related activities.

In an embodiment, the health band 101 may be configured to display information associated with a challenge that the wearer is participating in, such as a sports challenge associated with a minimum amount of exercise. The challenge may be private, public, may be sponsored and/or may be a function of an organization, such as a club. Information associated with a respective challenge level, a particular sport associated with a challenge, or other information associated with a challenge may also be displayed. For example, as the wearer is participating in an activity, substantially real-time feedback is provided representing the wearer's progress and/or improvement in ranking of participants may be provided.

The health band 101 can be worn during periods of exercise or strenuous activity and can be, therefore, constructed to be durable and resistant to moisture, breakage or the like. For example, the display portion 302 may be water resistant, shatter resistant, shock resistant or the like to ensure consistent and reliable operation. In one or more implementations, the display portion 302 includes E-INK technology, for low-power and high resolution display capability. Other suitable display technologies may be employed as well, such as organic light emitting diode (OLEO) displays, thin film transistor liquid crystal displays and plasma displays.

Also shown in FIG. 3 is a mode control button 304, which appears in the example in FIG. 3 as generally heart-shaped. When pressed, the mode control button 304 causes instructions to be processed for display portion 302 to change display modes. For example, after the mode control button 304 is pressed, the display portion 302 may change from displaying a wearer's health score to displaying the wearer's current heart rate. The mode control button 304 may instruct the health band 101 to operate in different modes depending upon, for example, the length of time that the mode control button 304 is pressed or the number of times that the mode control button 304 is pressed. In one or more other implementations, the heart-shaped portion of the health band 101 may perform no functionality other than to be a logo or other identifier of source. In one or more implementations, the health band 101 may be configured to be water proof, which may include elimination of mode control button 304.

In one or more implementations, the health band 101 includes a clasp 306 that closes on the strap. In one or more implementations, the health band 101 strap is sizable to fit comfortably on virtually any person, such as on a wearer's wrist. In the example shown in FIG. 3, the strap is configured with perforated sections 308 that may be used to size the health band 101 comfortably for a wearer, and to provide guides to cut off the excess portion of the strap. The strap may be sized so that it fits within the clasp 306, thereby providing a clean, finished and appealing appearance of the strap and that is customized in size for each respective wearer. In use, for example, the fitting of the strap enables sensors or other components provided with the health band 101 to operate effectively, for example, to sense biological and/or physiological characteristics of the wearer. The wearer may press a button or other suitable portion of the clasp 306, lift open a portion of the clasp 306 to release the strap, pull the strap out through the clasp 306 and, thereafter, cut the strap one perforated section 308 at a time until the health band 101 fits comfortably. Thereafter, the user may place the strap back in the clasp 306, press down on a cover portion of the clasp to lock the clasp and hold the strap in place. The strap of the health band 101 may be constructed of a soft, pliable material, such as rubber or soft silicon. In one of more other implementations, the health band 101 may include leather, stones (e.g., semi-precious or precious stones), logos or other artwork.

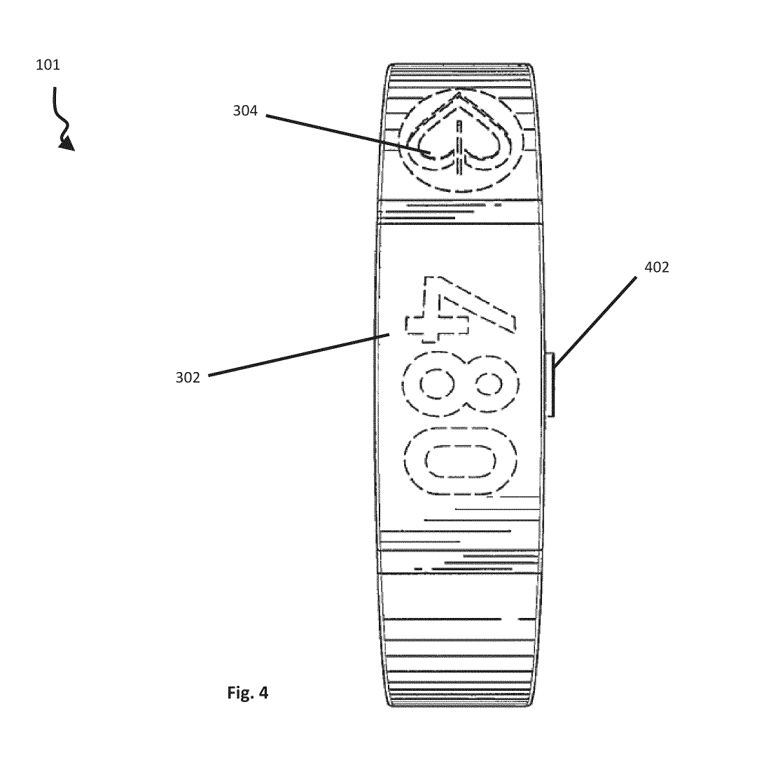

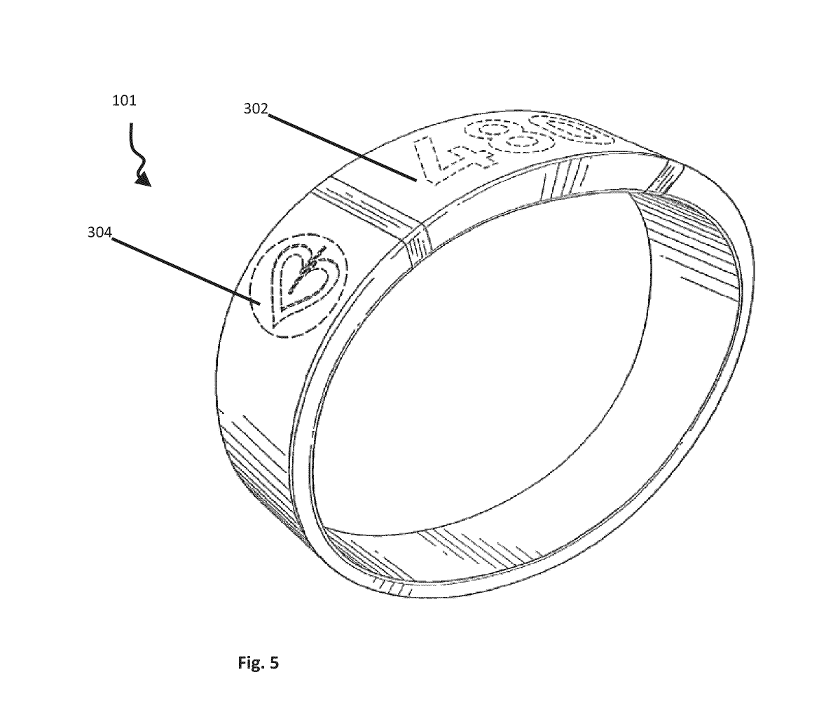

FIG. 4 illustrates a front elevation view of an example implementation of the health band 101. In the example shown in FIG. 4, the health band 101 is positioned and shown to include a control button 402, which may be useable for various functions, depending upon one or more implementations. For example, the display portion 302 may be configured to remain in an “always on” state and continuously display information, such as the wearer's current health score. Once pressed, control button 402 may cause instructions to be executed that cause display portion 302 to turn off. Control button 402 may be similarly used, for example, to turn on display portion 302.

In addition to turning display portion 302 on and/or off, other functionality may be associated with control button 402. For example, display portion 302 may be configured to display information without light, such as backlighting and, when pressed, control button 402 may cause instructions to be executed to activate LED or other backlighting near or within the display portion 302. In yet another configuration, control button 402, when pressed, may cause instructions to be executed to invert, rotate or otherwise modify the orientation of information displayed on the display portion 302. For example, a right-handed wearer of the health band 101 may prefer the orientation of information shown on display portion to be one way, while a left-handed wearer of the health band 101 may prefer a different (e.g., inverted) orientation. In yet another implementation, the orientation of information displayed on the display portion 302 may change depending upon orientation of the health band 101. For example, health band 101 may be configured with an accelerometer or other suitable technology that cause substantially automatic rotation of displayed information on the display portion 302. In one or more implementations, the health band 101 is configured with a three-axis accelerometer. Moreover, in one or more embodiments, the orientation of the display portion 302 of the health band 101 may alternate to be vertical, horizontal, diagonal or other orientation, to suit the wearer's particular taste and preference.

In one or more implementations, the accelerometer provided with the health band 101 provides various forms of functionality. Functionality that can be provided via the accelerometer can be associated with the user interface. For example, the wearer double taps the health band 101 and the accelerometer detects the movement and provides information via the user interface. Other functionality that can be provided via the accelerometer includes step counting (e.g., in accordance with pedometer functionality), and sleep monitoring.

For example and in connection with sleep monitoring, movement that occurs by the wearer is measured (e.g., counted) within a fixed time interval, for example, 15 minutes or less. These data can be continuously recorded, e.g., 24 hours a day/7 days a week, independent of whether the wearer is in bed and/or attempting to sleep. Moreover, an algorithm may be employed that uses various inputs to determine times when the wearer is (or has been) in bed and determines the amount of time when the wearer was sleeping and/or awake. Inputs to the algorithm can include, for example, the wearer's movement, the number of steps taken during a period of time (e.g., an hour), and the time of day.

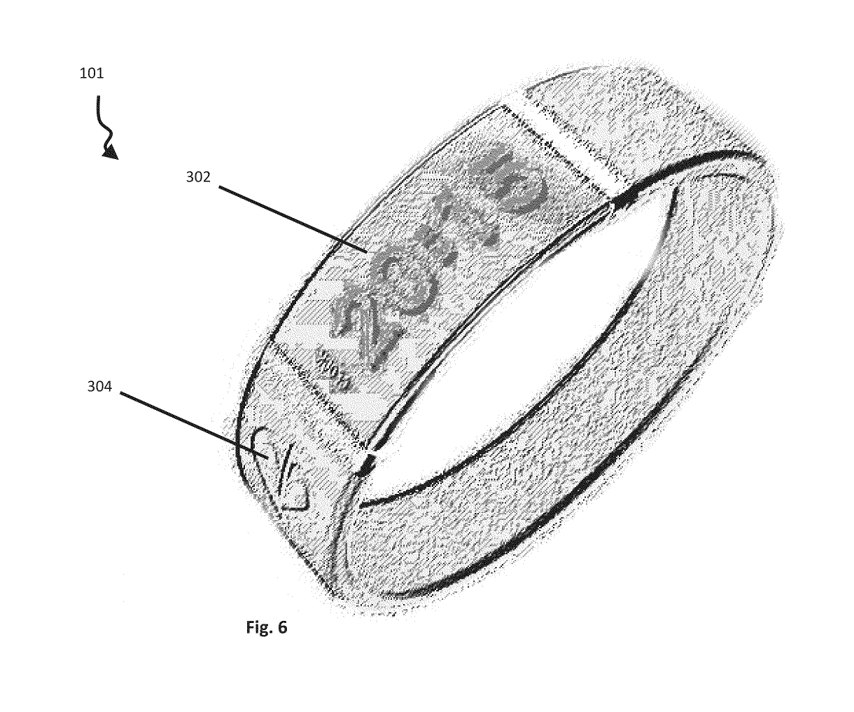

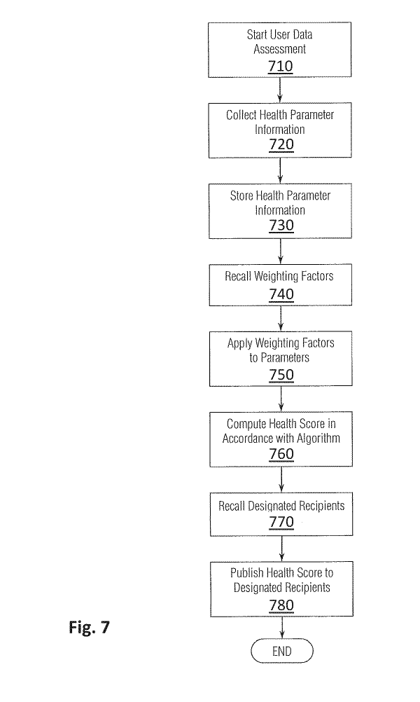

FIGS. 5 and 6 show additional perspective views illustrating implementation(s) of the health band 101 in accordance with example implementations of the present application, which do not include the clasp portion 306. In the example health band 101 shown in FIG. 7, the strap may be elastic to fit a plurality of variably sized wearers. Alternatively, the strap may be sized for wearers, for example, having particularly sized wrists. Also in the example shown in FIG. 6, the health hand 101 is operating, in a mode such that display portion 302 displays the time of day (e.g., 20:15), and demonstrates the multi-mode operability of the health band 101.

The health band 101 is configured to communicate with one or more computing devices and to display information for the wearer, such as associated with the wearer's health score. The health band 101 may be durable to withstand moisture, shock, and strenuous activity. Moreover, in one or more implementations the strap of the health band 101 is sizable. In one or more implementations, the electronics inside and/or associated with the health band 101 operate without a requirement for a cable for recharging a battery. Instead, a magnet or other suitable component is employed for recharging a battery provided with the health band 101. Alternatively, the health band 101 may operate by conventional battery, by solar cell, or be recharged, such as via a universal serial bus cable plugged into a computing device or other suitable power source.

Thus, as shown and described herein, the present application regards a system and method for efficiently and remotely obtaining, processing, sending, receiving and displaying information, including health-related information, on a health band. By providing such information on a wearable band, the information is current, accurate and may be useable in many different contexts, such as in hospitals, nursing care facilities, retirement communities or the like. The health band 101 of the present application may be configured to display information representing a person's daily movements, such as walking or other activity. It is recognized by the inventor that people, particularly elderly people, benefit from regular physical movement and activity, and the health band 101 may be configured to provide people with information associated with such activity.

In addition to displaying or otherwise providing information representing the wearer's activity, as well as biological and/or physiological information associated with the wearer, the health band 401 can be configured to provide reminders and/or notifications to the wearer. For example, reminders can be provided that are associated with adherence to medication, behavior activity or abstaining from certain activity), or for monitoring one or more medical conditions. In addition to displaying information via display portion 302, the health band 101 may be configured with a vibration mechanism (as known in the art) or other suitable configuration to provide an alert to a user. The health band 101 can vibrate to alert the user, for example, to take medication (e.g., a beta blocker, diabetes II medicine, blood pressure medication, or the like). Alternatively, the alert may remind the user to take some action, such as to draw blood to check blood glucose levels, to check heart rate or blood pressure, or to take some other action, such as to exercise e.g., take a walk or participate in a challenge), or to consume food (or stop consuming food). Thus, the health band 101 is configurable to issue alerts and/or reminders to users, such as by gently vibrating or in other suitable way (e.g., by emitting a mild electrical stimulation).

As noted herein, the health band 101 can be configured to provide audio information. In one or more implementations, one or more speakers and audio components can be included with the health band 101 to provide audio-based information. In addition, a microphone can be provided to receive Voice commands and/or audio input. Moreover, the health band 101 can be configured with a camera for capturing still and/or moving images. The ability to send and receive multimedia content (e.g., audio and/or visual content) provides the health band 101 with additional functionality associated with, for example, the ability to interact with others in various ways. For example, the health band 101 can be usable for remote communications, e.g., for users to communicate over a communications network. In one or more implementations, notifications associated with a health score, as described herein, are provided for users to compare with one or more other participants, including public figures (e.g., celebrities, sports figures or the like), and information can be broadcasted to and from the health band 101, substantially in real-time. Users can be provided with various kinds of challenge data, including from the people associated with one or more challenges. In one or more other implementations, numerical challenge information can be averaged with a particular geography (e.g., a town) or demographic, or a single individual whom the wearer is tracking against. This form of interactivity provides powerful and useful functionality for one or more wearers of the health band 101.

In addition, the health band 101 is usable to provide information associated with exercise and other workouts. Information, such as kilometer times, significant changes in heart rate, or guided training information, such as interval trainings, can be provided to a user substantially in real-time. Information may be displayed, and/or provided as multimedia-content. The health band 101 is well-suited as being configured to be worn by the user, such as on the wrist.

Furthermore and in one or more implementations, the health band 101 may be configured to display information relating to various ailments, such as Diabetes II, hypertension, or the like.

By way of overview and introduction, the present invention is described in detail in connection with a distributed system in which data acquisition, data storage, and data processing are used to produce a numerical score as a basis for assessing the relative health of a user.

In one or more implementations, the present application includes a computer-based implementation for the collection of health related parameters of a user and a user interface for the display of data. The computer-based application is implemented via a microcontroller that includes a processor, a memory and code executing, therein so as to configure the processor to perform the functionality described herein. The memory is for storing data and instructions suitable for controlling the operation of the processor. An implementation of memory can include, by way of example and not limitation, a random access memory (RAM), a hard drive, or a read only memory (ROM). One of the components stored in the memory is a program. The program includes instructions that cause the processor to execute steps that implement the methods described herein. The program can be implemented as a single module or as a plurality of modules that operate in cooperation with one another. The program is contemplated as representing a software component that can be used in connection with an embodiment of the invention.

A communication subsystem can be provided for communicating information from the microprocessor to the user interface, such as an external device (e.g., handheld unit or a computer that is connected over a network to the communication subsystem). Information can be communicated by the communication subsystem in a variety of ways including Bluetooth, WiFi, WiMax, RF transmission, and so on. A number of different network topologies can be utilized in a conventional manner, such as wired, optical, 3G, 4G networks, and so on.

The communication subsystem can be part of a communicative electronic device including, by way of example, a smart phone or cellular telephone, a personal digital assistant (PDA), netbook, laptop computer, and so on. For instance, the communication subsystem can be directly connected through a device such as a smartphone such as an iPhone, Google Android Phone, BlackBerry, Microsoft Windows Mobile enabled phone, and so on, or a device such as a heart rate or blood pressure monitor (such as those manufactured by Withings SAS), weight measurement scales (such as those manufactured by Withings SAS), exercise equipment or the like. In each instance, the devices each comprise or interface with a module or unit for communication with the subsystem to allow information and control signals to flow between the subsystem and the external user interface device. In short, the communication sub-system can cooperate with a conventional communicative device, or can be part of a device that is dedicated to the purpose of communicating information processed by the microcontroller.

When a communicative electronic device such as the types noted above are used as an external user interface device, the display, processor, and memory of such devices can be used to process the health related information in order to provide a numerical assessment. Otherwise, the system can include a display and a memory that are associated with the external device and used to support data communication in real-time or otherwise. More generally, the system includes a user interface which can be implemented, in part, by software modules executing in the processor of the microcontroller or under control of the external device. In part, the user interface can also include an output device such as a display (e.g., the display).

Alternatively or in addition, the user can self-report his or her health related information by manually inputting the data into the health band 101. Thus, in another implementation health related data of a person is entered directly (or indirectly) into the health band 101 and, thereafter, provided across a network to information processor 102. (All computers described herein have at least one processor and a memory.)

Regardless of the implementation, the system provides a means for assigning a numerical value that represents the relative health of an individual. The numerical value is described herein as a “health score” and can be used to assess to the individual's health based on health related information collected from a user. The health score is calculated based on the collected health information using an algorithm. The user or the communication subsystem provides the system the health related information concerning a number of health parameters. Predetermined weighting factors are used to assign a relative value of each of the parameters that are used to calculate the health score. The user's health score is then calculated by combining the weighted parameters in accordance with an algorithm. For example, the parameters can be a person's blood glucose level and body weight. A weighting factor “a” is applied to the blood glucose data and a weight factor “b” can be applied to the body weight data. If the blood glucose data is a more important factor in determining a person's health than body weight, then the weighting factor “a” will be larger than weighting factor “b” so that the blood glucose data has a larger impact on the calculated health score (e.g., Healthscore=Glucose*a+(Weight/100)*b). In certain implementations, the weighting factor is a non-unity value (e.g., greater or less than one, but not one). Fewer or additional factors can be included in the calculation of the health score, and an offset value can be included that is added or subtracted or which modifies the entire calculation, in certain implementations such as to account for age or gender as two possible reasons; however, the foregoing is intended as a non-limiting example of how to calculate a health score. Other parameters that can be measured and included in the calculation include blood pressure measurements, height, body mass index, fat mass, medical conditions such as diabetes, ventricular hypertrophy, hypertension, irregular heartbeat and fasting glucose values. Where absent, a parameter can be omitted from the calculation or it can be estimated from other parameters and/or values obtained from a sample group of individuals having similar parameters.

In addition to intrinsic medical parameters, physical activity of a user is also taken into account when calculating his or her health score. As noted herein, physical activity can be monitored by the health band 101 via an appropriate sensor dependent on the activity. Sensors can include a GPS unit, an altimeter, a depth meter, a pedometer, a cadence sensor, a velocity sensor, a heart rate monitor or the like. In the case of gym-based activities, computerized exercise equipment can be configured to provide data directly on the program completed by the user (for example, a so-called elliptical/cross-trainer can provide far better data on the workout than a user's pedometer etc). Although automated capture of parameters concerning a user's physical activity is preferred, a user interface for manual activity entry is also provided. In this regard, an exercise machine such as a treadmill, elliptical, stationary bike or weight lifting machine with a rack of weights or bands can be provided with a communications interface to communicate with the system described herein to provide extrinsic physical activity parameters to the system and to receive and further include a processor configured to process data from the system so as to automatically adjust an exercise program at the exercise machine to meet a goal, challenge, or other objective for that user.

Lifestyle data such as diet, smoking, alcohol consumed and the like can also be collected and used in calculating the health score. In one embodiment, a barcode or RFID scanner can be used by a user to capture data on food and/or nutrition that is consumed, and that is then translated at a remote system, such as the server or a website in communication with the server, into parameters such as daily calorie, fat and salt intake. In part, the system relies on such data being provided by the user while other data can be obtained through data network connections once permissions and connectivity rights are in place.

Physical activity and lifestyle data is tracked over tune and a decay algorithm is applied when calculating its effect on the health score, as is discussed in more detail below. As such physical activity far in the past has a reduced positive effect on the health score. Preferably, the weighting factors used in the algorithm for the computation of the health score are adjusted over time in accordance with a decay component which is arranged to reduce the relative weight of the parameters that are used in the calculation. The decay component can itself comprise a weighting value, but can also comprise an equation that takes into account at least one factor associated specifically with the user, such as the user's weight or weight range, age or age range, any medical conditions known to the system, and any of the other parameters that may be known to the system, or a curve that is configured in view of these factors so that a value can be read from the curve as a function of the values along the axes for that user. In this way, the decay component can reduce the relative weight of the parameters used in the health score calculation for a first user differently than for another user, such as when the first user has a first age or age range and the second user has a second age or age range.

A central system, preferably a database and website that can be hosted, for example, by the information processor 102, maintains data on each user and his or her health score and associated parameters and their trends over time. The data can be maintained in such a way that sensitive data is stored independent of human identities, as understood in the art.

The calculated health score for each user is then processed in dependence on a system, group or user profile at the central system. Depending on the profile settings, the health score and trends associated can cause various automated actions. For example, it can cause: triggering of an automated alert; providing user feedback such as a daily email update; triggering the communication of automated motivation, warnings and/or goal setting selected to alleviate a perceived issue; adjustment of a training program; or automated referral for medical analysis.

The user's health score is also provided to a designated group of recipients via a communication portal. The group of recipients can comprise selected, other users of the system (e.g., friends and family) so that the health scores of the selected, other users can be compared against the health score of still others. In alternative arrangements, all users can see other user's scores, or the group of recipients can be defined as a specific health insurance provider so that price quotes can be provided to insure the individual. Other possibilities are within the scope the invention.

Referring now to FIG. 7, a schematic flow diagram according to one embodiment of the invention is described in support of an assessment of a person (e.g., a patient or user) to provide a health score. At step 710, the user initiates the process for the collection, processing, and publishing of health related data. For example, a person using a mobile electronic device (e.g. a smart phone or portable computing device) selects the software application, which starts the program running on the device processor, or the user can access an Internet based web page in which code is executed on a remote processor and served to the user's local device. An identification module prompts the user to identify him and authenticate his identity. This can be accomplished by prompting the user to enter a user name and password, or by other means, such as a fingerprint reader, keyfob, encryption or other mechanism to ensure that identity of the user. Alternatively, if the user is accessing the system via a personal electronic device, identification data can be stored in the local device memory and automatically accessed in order to automatically confirm the identity of the user.

At step 720, a data collection module executing on the processor can prompt the user to provide health related data corresponding to a number of parameters. In one implementation, one or more the parameters are provided automatically by the communication subsystem. The parameters can include the user's body weight, height, and age and fitness activity information. Such measurable medical parameters are intrinsic parameters of the user. The user's body weight and height provide information about the user's current state of health. The fitness activity information corresponds to the amount of exercise the user engages in. This information is an example of a physically activity parameter that is an extrinsic parameter of the user. For example, the user can enter information about his or her daily fitness activities, such as the amount of time the user engaged in physical activity and the type of physical activity. If the user went to the gym and exercised on a bicycle for thirty minutes, for example, that information is entered into the system. The user's fitness activity information provides information about the actions that are being taken by the user in order to improve his or her fitness.

A user's body weight, height, age and fitness activity information are just some of the parameters for which information can be collected. The system can collect and process a multitude of other parameters that can be indicative of a user's health. For example, parameters can include blood glucose levels, blood pressure, blood chemistry data (e.g., hormone levels, essential vitamin and mineral levels, etc.), cholesterol levels, immunization data, pulse, blood oxygen content, information concerning food consumed (e.g., calorie, fat, fiber, sodium content), body temperature, which are just some of a few possible, non-limiting examples of parameters that can be collected. Various other parameters that are indicative of a person's health that can be reliably measured could be used to calculate a person's health score.

The collected health parameter information is stored in a memory at step 730. At step 740, a weighting module recalls weighting factors from the memory. The weighting factors can be multiplication coefficients that are used to increase or decrease the relative value of each health parameters. A weighting factor is assigned to each health parameter as shown in the formulas herein. The weighting factors are used to control the relative values of the health parameters. Some health parameters are more important than others in the calculation of the users' health score. Accordingly, weighting factors are applied to the health parameters increase or decrease the relative affect each factor has in the calculation of the user's health score. For example, a user's current body weight can be more important than the amount of fitness activity the user engages in. In this example, the body weight parameter would be weighted more heavily by assigning a larger weighting factor to this parameter. At step 750, the weighting module applies the recalled weighting factors to the collected health parameter values to provide weighted health parameter values. The weighting factor can be zero in which case a particular parameter has no impact on the health score. The weighting factor can be a negative value for use in some algorithms.

After the parameters have been weighted, the user's health score is computed at step 760 via a scoring module operating in the processor. The scoring module combines the weighted parameters according to an algorithm in one implementation, the health score is the average of the user's body mass index (BMI) health score and the user's fitness health score minus two times the number of years a person is younger than 95. The algorithm formula for this example is reproduced below:

Health Score=((BMI Healthscore+Fitness Healthscore)/2)−2*(95−Age).

The user's BMI Healthscore is a value between 0 and 1000. The BMI Healthscore is based on the user's BMI, which is calculated based on the user's weight and height, and how much the user's BMI deviates from what is considered a healthy BMI. A chart or formula can be used to normalize the user's BMI information so that dissimilar information can be combined. A target BMI value is selected which is assigned a maximum point value (e.g. 1000). The more the user's BMI deviates from the target value the fewer points are awarded. The user's Fitness Healthscore is based on the physical activity or exercise of a person. In one embodiment, it is the sum of the number of fitness hours (i.e., the amount of time the user engaged in fitness activities) in the past 365 days where each hour is linearly aged over that time so that less recent activity is valued less. The resulting sum is multiplied by two and is capped at 1000. This normalized the fitness information so that it can be combined to arrive at the health score. A target daily average of fitness activity is selected and is awarded the maximum amount of points (e.g. 1000). The user is awarded fewer points based on how much less exercise that is engaged in compared to the target.

In another implementation, the health score is determined from a number of sub-scores that are maintained in parallel beyond the BMI health score and the fitness health score. Likewise, the health score can be determined using similar information in a combinative algorithm as discussed above using different or no age adjustments.

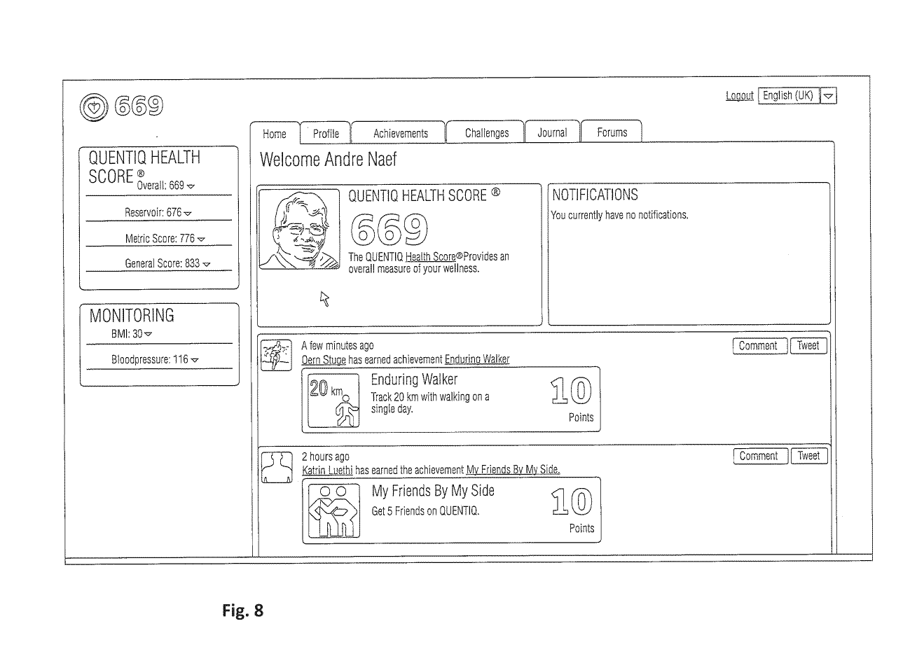

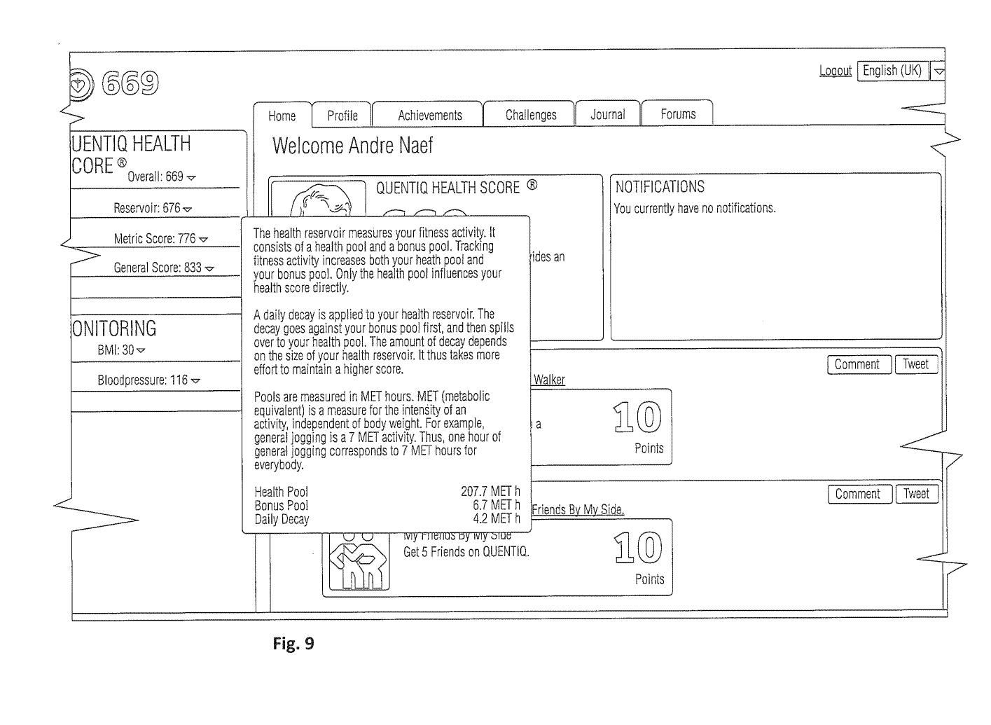

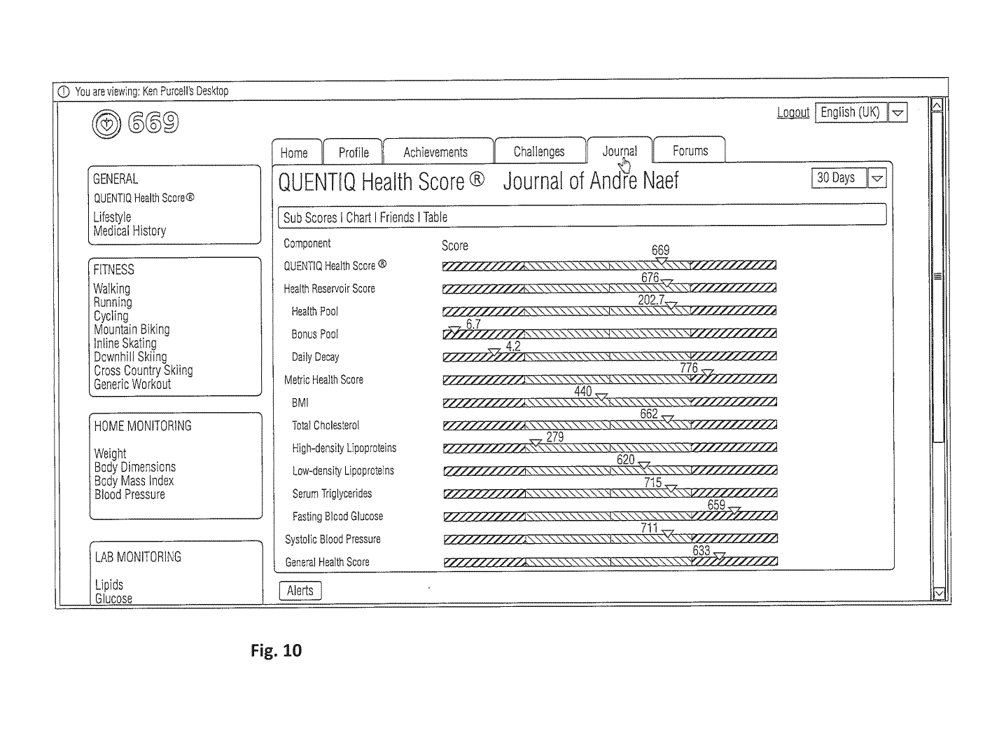

Intrinsic medical parameters are processed to determine a base health score. Extrinsic parameters such as those from physical exercise are processed to determine a value that is allocated to a health pool and a bonus pool. The value, preferably expressed in MET hours, associated with a physical activity is added to both the health pool and the bonus pool. A daily decay factor is applied to the bonus pool. Any excess decay that cannot be accommodated by the bonus pool is then deducted from the health pool. The amount of decay is determined dependent on the size of the health pool and bonus pool such that a greater effort is required to maintain a high health and bonus pool. The health pool value is processed in combination with the score from the intrinsic medical parameters in order to calculate the overall health score value. This can be on a similar basis to the earlier described implementation or it can include different parameters and weighting factors. In one embodiment, the health pool value is a logarithm or other statistical function is applied to age the respective values over time such that only the most recent activity is counted as being fully effective to the health/bonus pool. An example user interface showing the health score, the health reservoir and selected other measured parameters (as it will be appreciated that many simply combine to make up the scores) is shown in FIGS. 8 and 9. Various sub-scores and their trends are recorded, as is shown in FIG. 10.

As will be appreciated, MET hours are kcal expended divided by kilograms of body weight, i.e. 100 kcal expended by a person of 50 kg is 2 MET h. This is “normalized energy”, making the system fair for persons of all weights. With this method, pools can be the same size for each per person as energy is normalized for the person based on his or her body weight.

In one implementation, each person is assigned a health pool having a capacity of 300 MET h and a bonus pool having a capacity of 60 MET h.

When someone performs activity A, the pools are updated as follows:

H=min(H+A*alpha, 300)

B=min(B+A*(1−alpha), 60)

Where H is the health pool score, B is the bonus pool score, A is the MET h value for the activity and alpha is a system wide constant (selected between 0 and 1) that determines the proportion in which the activity contributes to the respective pools.

The activity is split between the health pool and the bonus pool. Any excess MET h activity going over the cap of any pool is discarded. A daily decay value D is applied to the pools as follows:

D=f(H, B)

B=B−D

If B<0;

D=D+B

B=0

If D<0;

D=0

The decay is fully applied to the bonus pool, and if the bonus pool is empty, the remainder is applied to the health pool. In this embodiment, no pool ever goes below zero.

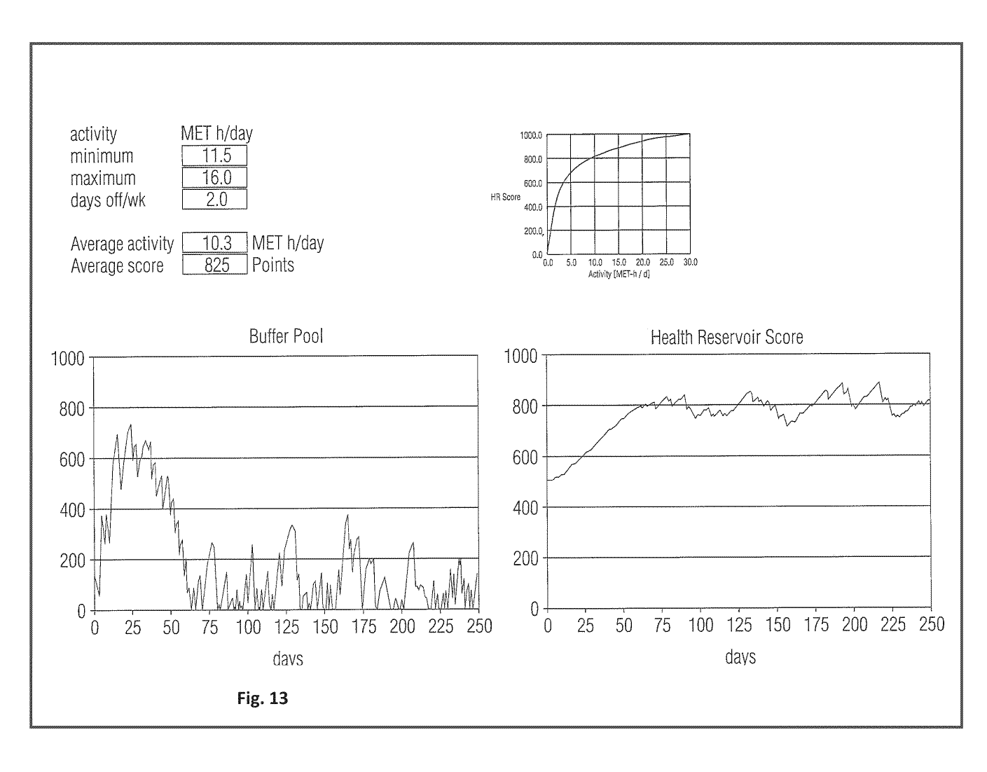

The system finds its equilibrium where A equals f(H, B), i.e. where the average daily activity matches the average daily decay. The function f(H, B) is highly non-linear with regard to H and B. In essence, it takes sub-linearly less effort to maintain a small pool, and super-linearly more effort to maintain a large pool. This is to make sure that the average person can maintain a, say, half-full health pool (150, corresponding to a score of 500), whereas it takes a massively higher effort (typically only delivered by a professional endurance athlete) to maintain a full health pool (300, corresponding to a score of 1000). FIG. 13 shows a simulation of the buffer pool and health reservoir score over time assuming activity varying between 11.5 and 16 MET h per day and 2 days off per week. A perfect health reservoir score of 1000 would require 30 MET h activity per day, as can be seen from the curve in the top right corner of FIG. 13.

Preferably, the health score is based on a weighted combination of health factor(s) and the exercise record of the person over time. The health factors can be updated regularly by the user. For example, the user can provide health related information after every event that is tracked and processed by the system. The user can update after a meal, after exercising, after weighing himself, etc. In the case of recordal of an activity/event by a sensor, portable device or the like, the captured/calculated parameters can be automatically uploaded and used to produce a revised health score. For example, feedback could be provided showing the effect of exercise while a user is running, working out on exercise equipment etc. In selected embodiments, feedback can be provided to an administrator such as a gym staff member where it is determined that a user is exceeding a predetermined threshold (which due to knowledge of their health can be varied respective to their health score or other recorded data). Accordingly, the health related data can be updated in a near real-time manner.

The user can also update the information twice daily, once daily, or at other periodic times. Moreover, the health score can be based on an average of the information over time. Fitness activity, for example, can be averaged over a period of time (e.g. over a week, month, or year). Averaging data over time will reduce the impact to the health score caused by fluctuations in data. Periods in which the data was uncharacteristically high (e.g., the person was engaging large amount of fitness activity over a short period of time) or uncharacteristically low (e.g., person engaged in no fitness activity for a week due to an illness) does not dramatically affect the health score with averaging over time. The health related information can be stored in the memory or in a database accessible by the processor.

The stored data can also be used to predict future health scores for a user. A prediction module can analyze past data (e.g., fitness habits, eating habits, etc.) to extrapolate a predicted health score based on an assumption that the user will continue to act in a predicable manner. For example, if the data shows that a user has exercised one hour every day for the past thirty days, the prediction module can predict, in accordance with a prediction algorithm, that the user will continue to exercise one hour for each of the next three days. Accordingly, the scoring module can calculate a predicted health score at the end of the next three days based on the information from the prediction module. It can also factor the prediction into other actions. For example, the system can suggest a more exerting physical activity level or challenge to someone who has a high health score but is predicted based on past experience to then take a number of days off for recuperation. Furthermore, the system can provide encouragement to the user to maintain a course of activity or modify behavior. For example, the system can send a message to the user indicating that if the user increased fitness activity by a certain amount of time, the health score would go up by a certain amount. This would allow the user set goals to improve health.

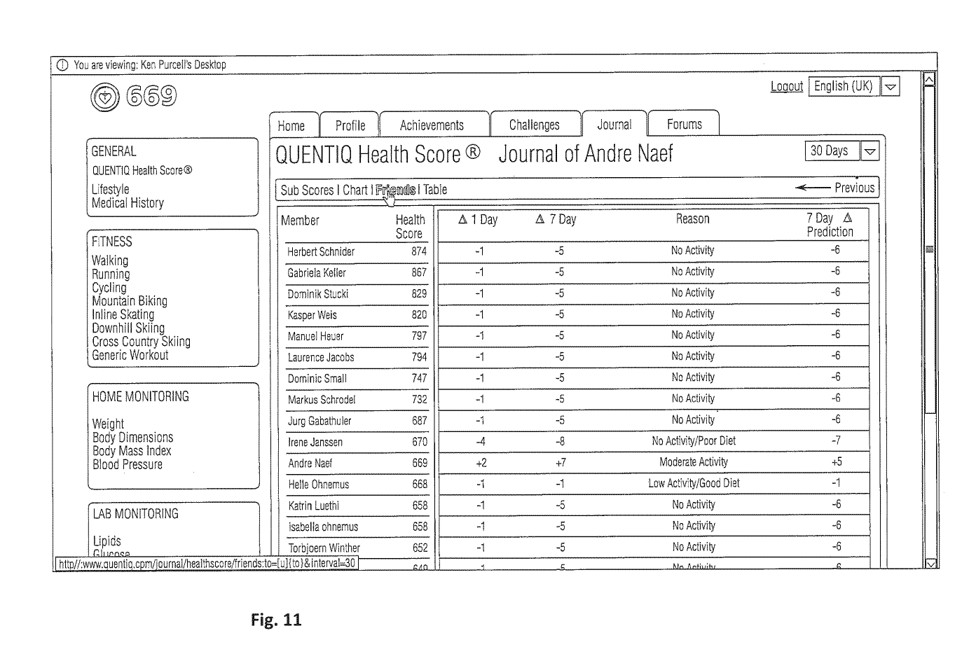

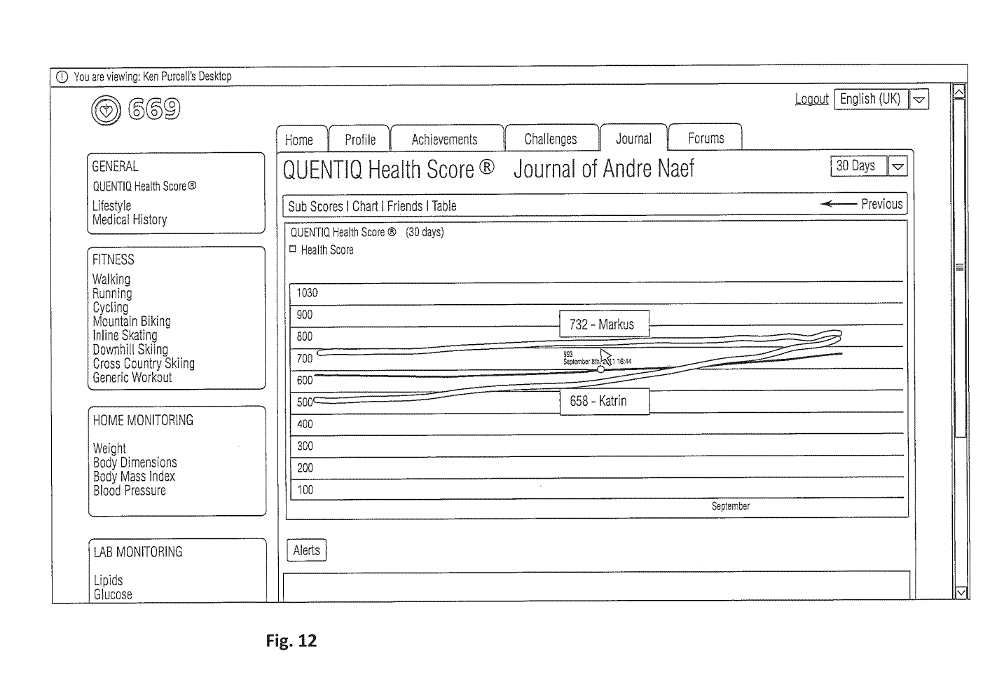

The use of the health score allows for a relative comparison of a user's health with that of another person's even though each person can have very different characteristics, which would make a direct comparison difficult. For example, a first user (User 1) can have a very different body composition or engage in very different fitness activities as compared to a second user (User 2), which makes direct comparison of the relative health of each user difficult. The use of the health score makes comparison of the two users possible with relative ease. In one example, User 1 is slightly overweight, which would tend to lower User 1's health score. However, User 1 also engages is large amounts of fitness activities, thereby raising the user's overall health score. In contrast, User 2 has an ideal body weight, which would contribute to a high health score, but engages in very little fitness activity, thereby lowering the health score. User 1 and User 2 are very different in terms of their health related parameters. Accordingly, it would be very difficult to assess and compare the relative health of User 1 and User 2. In accordance with the invention, information related to certain health parameters is collected from User 1 and User 2, which is used to calculate an overall health score. A comparison of User 1's and User 2's health score allows for an easy assessment and comparison of the health of these two users even though they are very different and have very different habits. Therefore, the health score has significant value so that members of a group can compare their relative health and so that other entities (e.g., employers, health care insurers) can assess the health of an individual. Examples are shown in FIGS. 11 and 12 in which tabular (current) and graphical (historic, current and predicted) scores of different users are shown. As can be seen in FIG. 12, Katrin is expected to surpass the user (Andre) shortly unless he improves his lifestyle and performance. In FIG. 11, the impact of the decay algorithm is illustrated to show the effect on the health score of a given user (Andre) and the people he has identified as friends. As noted user Andre has a current health score of 669 which situates this user between friends Irene (health score 670) and Helle (health score 668). The decay algorithm has acted on all of the health scores shown in the screen shot of FIG. 11, as indicated in the “? 1 Day” column. More particularly, most of the friends of Andre have had their health score reduced by 1 point due to the reason of “no activity.” A lack of data input to the system is a basis for the processor executing the decay algorithm to determine a “no activity” status for a given user. The one day effect of this status according to the illustrated decay algorithm for most of the users is a reduction of 1 point in one day, and a reduction of 5 points over the course of a week. As such, the decay algorithm can have a tapering, non-linear impact on an overall health score.

As illustrated, user Andre has had moderate activity registered into a memory that is accessible to the system. As a result, the moderate activity is processed and results in a one day change (delta) that is positive, and a change that counteracts the influence of the decay algorithm. Consequently, Andre will be able to observe, as well as the friends that have access to his published health score, that he increased his score from 667 to 669 in one day, and from 662 to its present value over the past seven days as a result of “moderate activity.” Moreover, a prediction is computed using the underlying algorithm and an extrapolation of data based on most recent reasons (that is, received data) to increase another 5 points. On the other hand, due to low activity, but a good diet, Helle in the same time period went down 1 point in the last day and a total of point in the last 7 days and is predicted to lose another point if this rate continues. As such, Helle is provided with feedback by execution of the algorithm and the outputs provided by the system which can encourage more activity. On the other hand, Irene has no activity and a poor diet which results in a more aggressive change to her current health score and the longer-view historical and predicted impact on her score. Again, this feedback, which can be provided to users and their friends or to members of a group of users that have associated together for a challenge, etc. to provide individual or team motivation to engage in fitness activities, eat well, and so on.

Moreover, the health score provides an indication of the relative health of the individual without revealing the underlying data used to calculate the health score, which can be sensitive information. For example, a user may be uncomfortable revealing his or her weight, age, or amount of time they spend exercising to others persons or entities. Persons can be embarrassed to share his or her weight or the fact that they virtually never go to the gym. However, since the health score is derived from several factors, the underlying data used to calculate the score is kept private. This feature will facilitate the sharing of the user's overall health because users will not have to disclose private data about themselves. For example, a person may be slightly overweight, but she goes to the gym often. Accordingly, that person can receive a relatively good health score. While the person may not want to disclose her weight, she can still disclose her health score which conveys information about her relative health without disclosing the underlying details. The intrinsic medical parameters (e.g. weight, height, etc.) and the extrinsic physical activity parameters (e.g. exercise duration, frequency, intensity, etc.) are transformed into a masked composite numerical value. The masked numerical value is published while the collected information concerning the intrinsic medical parameters and extrinsic physical activity parameters is maintained private. The underlying intrinsic medical parameters and extrinsic physical activity parameters are protected so that a third party is not able to determine those parameters based on the health score number. This is because the parameters can vary in many different ways and yet the health score number could be the same (e.g., a heavier person that exercises frequently can have the same health score as a person that is not overweight but does not exercise as frequently). Thus, having the health score alone does not reveal a person's health related parameters. Accordingly, the underlying health statistics are masked, yet the health score can be used as a benchmark to indicate a person's health for a variety of applications.

After the scoring module calculates the health score of the user, at step 770, a publication module recalls from the memory the designated group of recipients that are authorized to receive the health score. The group of recipients can be friends or family of the user, sports teammates, employers, insurers, etc. At step 780, the publication module causes the health score to be published to the designated group. In the case that the information is to be published to a group of friends, the information can be published to a social networking interne based portal in which access to the data is limited to those designated members of the group.

The health parameter data and health scores can be stored over time, in a memory or other database, so that a user can track his or her progress. Charts can be generated in order for a user to track progress and analyze where there can be improvement in behavior. Moreover, trends can be identified that can lead to the diagnosis of medical problems and/or eating habits. For example, if a person's weight is continuing to increase despite the same or increased amount of fitness activity, the system can trigger or suggest that they seek certain medical tests (e.g. a thyroid test, pregnancy test) to determine the cause of the weight gain.

In certain implementations, the majority of the system is hosted remotely from the user and the user accesses the system via a local user interface device. For example the system can be internet based and the user interacts with a local user interface device (e.g., personal computer or mobile electronic device) that is connected to the interne (e.g., via a wire/wireless communication network) in order to communicate data with the internet based system. The user uses the local interface device to access the interact based system in which the memory and software modules are operating remotely and communicating over the interact with the local device. The local device is used to communicate data to the remote processor and memory, in which the data is remotely stored, processed, transformed into a health score, and then provided to the designated groups via a restricted access internet portal. Alternatively, the system can be primarily implemented via a local device in which the data is locally stored, processed, and transformed into a health score, which is then communicated to a data sharing, portal for remote publication to the designated groups.

The system can be implemented in the form of a social networking framework that is executed by software modules stored in memory and operating on processors. The system can be implemented as a separate, stand alone “health themed” social networking system or as an application that is integrated with an already existing social networking system (e.g., Facebook, MySpace, etc.), The user is provided with a homepage in which the user can enter information. manage which information is published to designated groups, and manage the membership of the designated groups. The homepage includes prompts to the user to enter the health related information for the each of the various parameters. The user can enter his or her weight, date of birth, height, fitness activity, and other health related information. The user's health score is then calculated. The health score is shared with other users that are designated as part of a group permitted to have access to that information. Moreover, the user can view the health score information of others in the group. Accordingly, the user is able to compare his or her overall health with the health of others in the group. Comparison of health scores with others in the group can provide motivation to the individuals in the group to compete to improve their health scores. Other information, such as health tips, medical news, drug information, local fitness events, health services, advertising and discounts for medical and/or fitness related supplies and service, issuance of fitness challenges or health related goals, for example, can be provided via the homepage.

In further implementations, the health score can be a composite of a Metric Health Model score and a Quality of Life Model score. Combining scores from multiple models provides a more holistic assessment of a user's health. The Metric Health Model score assesses a user's health based on relatively easily quantifiable parameters (e.g., age, sex, weight, etc,) and compares those numbers to acceptable populations study models. The Quality of Life Model score focus on a user's self-assessed quality of life measure based on responses to a questionnaire (i.e., the system takes into account the user's own assessment of their health and life quality) because there are correlations between how an individual “feels” about his or her life and a realistic measure of health. A combination of the scores from these two models, which will be discussed in more detail below, provides a more inclusive and holistic assessment of health.

The Metric Health Model score is based on medical parameter information of a user, such as their medical history information, attributes, physiological metrics, and lifestyle information to the system. For example, the system can provide the user a questionnaire to prompt responses (yes/no, multiple choice, numerical input, etc.) or provide the user with form fields to complete. Medical history information can include the user's history of medical conditions and/or the prevalence of medical conditions in the user's family. Examples of medical history information can include information such as whether the user has diabetes, has direct family members with diabetes, whether the user or family members have a history of heart attack, angina, stroke, or Transient Ischemic Attack, a history of atrial fibrillation or irregular heartbeat, whether the user or family members have high blood pressure requiring treatment, whether the user or family members have hypothyroidism, rheumatoid arthritis, chronic kidney disease, liver failure, left ventricular hypertrophy, congestive heart failure, regular use of steroid tablets, etc.

The Metric Health Model score can also be based on user attributes. The attributes can include age, sex, ethnicity, height, weight, waist size, etc. In addition, Metric Health Model score can be based on physiological metrics of the user. Examples of physiological metrics can include systolic blood pressure, total serum cholesterol, high-density lipoprotein (HDL), low-density lipoprotein (LDL), triglycerides, high-sensitivity C-reactive protein, fasting blood glucose, etc. The inputs can also include parameters of a user's lifestyle. For example, lifestyle parameters can include inputs about whether the user is a smoker (ever smoked, currently smokes, level of smoking, etc.), how much exercise the user performs (frequency, intensity, type, etc.), type of diet (vegetarian, high-protein diet, low-fat diet, high-fiber diet, fast-food, restaurant, home cooking, processed and pre-packaged foods, size of meals, frequency of meals, etc.). These are some of the examples of parameters that can be used to compare the user's health indicators to survival probability models in order to calculate the user's Metric Health Model score.

Survival probability prediction models can be used to predict the probability that an individual will suffer one or more serious health events over a given time period. Mathematical models can estimate this probability from observed population characteristics. Using observational data on a set of unambiguous severe health events, such as stroke or cardiac infarction, models can generate the probability that an individual will suffer one such event over a given time horizon from a set of measurements of markers, or predictors, for the event (e.g., information about a user's medical history, attributes, physiological metrics, lifestyle, etc. as described above). The time distance between the moment the predictors are measured, and the target event that is generated by such models, is referred to as a survival probability, although it must be understood that not all target events considered are necessarily fatal.

These survival probability models are typically derived from the study of generally large populations that are followed for a considerable length of time, usually more than ten years, and the statistics collected on the observation of the target event(s) are summarized and generalized using mathematical methods. There are a number of such models that exist that have been extensively validated and maintained and improved by periodically updating the model's parameters using new data. Examples of existing models can include a subset of models developed and maintained by the Framingham Heart Study (an extensive bibliography on results obtained from the Framingham Heart study is available at www.framinghamheartstudy.org/biblio), a subset of the models developed and maintained by the University of Nottingham and the QResearch Organization (see, for example, J Hippisley-Cox et al, Predicting cardiovascular risk in England and Wales: prospective derivation and validation of QRISK2, BMJ 336:1475 doi: 10.1136/bmj.39609.449676.25 (Published 23 Jun. 2008)), the ASSIGN model developed by the University of Dundee (see, for example, H Tunstall-Pedoe et al, Comparison of the prediction by 27 different factors of coronary heart disease and death in men and women of the Scottish heart health study: cohort study; BMJ 1998;316:1881), the Reynolds model (see, for example, PM Ridker et al, C-Reactive Protein and Parental History Improve Global Cardiovascular Risk Prediction: The Reynolds Risk Score for Men, Circulation 2008;118;2243-2251, and Development and Validation of Improved Algorithms for the Assessment of Global Cardiovascular Risk in Women, JAMA, Feb. 14, 2007-Vol 297, No. 6), the PROCAM model from the Münster Heart Study (see, for example, Simple Scoring Scheme for Calculating the Risk of Acute Coronary Events Based on the 10-Year Follow-Up of the Prospective Cardiovascular Münster (PROCAM) Study, Circulation. 2002;105:310-315), and the SCORE model (see, for example, RM Conroy et al, Estimation of ten-year risk of fatal cardiovascular disease in Europe: the SCORE project, European Heart Journal (2003) 24, 987-1003). Other constituent risk models can also be included. In addition, precursor models can also be used. Precursor models predict the development of a first condition (e.g. high blood pressure), wherein the development of the first condition is predictive of developing a second condition (e.g., heart disease). There are models that generate estimates of the probability of developing diabetes or high blood pressure, for example, which are two important predictors of mortality. A high probability of developing diabetes in five years, for instance, will independently increase the probability of a serious cardiovascular event within the next ten years. Several such precursor models can be included and the inclusion of precursor models leads to more accurate metric risk models, but more importantly, also leads to the possible reduction of the risk of mortality through well-defined modifiable aspects of lifestyle.

Traditional survival probability models have certain inherent limitations that result from the procedures used to build them. In deriving such models, researchers compromise between accuracy and usability. It is difficult for an inductive model, meaning a model derived directly from data, to include all possible predictors. This is in part because not all relevant predictors of a particular event are known, but also in part because some known predictors may be difficult or expensive to measure. In fact, several well-known markers of risk, such as genetic factors, are often not included in such models. Therefore, several potential and known predictive metrics can be excluded as covariates when deriving a given survival model.

Survival probability models are built using data collected from a given population, and thus summarize and generalize morbidity and mortality characteristics of the studied population. However, such a model might be at variance when compared with risk estimates derived from other populations. When a given model is used in a population that differs from the one where the model was built it often underestimates or overestimates a particular risk because only a few predictors are often considered, and because other relevant predictors that may not be included in the model might very well differ between two populations.

Given the above discussion, together with basic probabilistic logic, a judicious combination of models derived for several different populations will generate a better view of the risks that an individual picked at random is exposed to, and will thus be more robust in estimating risks for the population at large. Furthermore, based on mathematical grounds, under very general assumptions, certain model combination methods, referred to as predictor boosting, can improve the accuracy of the constituent models. In fact, boosting, a set of models, when done correctly, will produce a model with accuracy that is, at worst, equal to that of the most accurate model in the boosted set.

Accordingly, the Metric Health Model score can be calculated by comparing the user's medical parameter information to the survival probability models. A score, preferably in the range of 0 to 1000, with the top end signifying perfect health and the low side signifying poor health, can be derived following a two-step process. First, an overall survival probability is obtained from a combination of the survival probabilities generated by individual survival probability models, as described above. Second, the resulting survival probability, which is a number in the 0 to 1 range, is transformed using a parametric nonlinear mapping function into the 0 to 1000 range. The parametric mapping function is tuned so that it is linear, with a high slope, in the region of typical survival probabilities, and asymptotically slopes off in the low and high ends of the survival probability distribution. The mapping function is designed to be strongly reactive to changes in the typical survival probability region.

As discussed above, the health score can be composed of the Metric Health Model score, and also the Quality of Life Model score. The Quality of Life Model score is based on a user's answers to a set of questionnaires. The system can include several different questionnaires with some questions in common. The type of questionnaires and the type of questions therein presented to the user can be tailored based on a user's health parameters (i.e., user age, other data in the user's medical history, etc.). A specific questionnaire can be generated and presented to the user on the basis of information on the user that is known to the system. The questions can be presented with an appropriate multiple choice response that the user can check/tick on a form, with no free-form text is entered by the user to permit easier assessment of the responses. Other types of responses are possible (e.g., rating how true a statement is to the user 1-10). The following list provides several sample questions (in no particular order) on a number of health-related quality of life topics that can be used in a system questionnaire.

### Sample Questions:

How do you rate your quality of life?
How do you rate your overall health?
How much do you enjoy life?
To what extent do you feel your life to be meaningful?
How well are you able to concentrate?
How safe do you feel in your daily life?
How healthy is your physical environment?
Are you satisfied with your appearance?
To what extent do you have the opportunity for leisure activities?
How much do you need any medical treatment to function in your daily life?
For how long have your activities been limited because of your major impairment or health problem?
Do you need help in handling your personal care needs because of health problems?
Do you need help in handling your routine needs because of health problems?
Are you limited in any way in any activities because of any major impairment or health problem?
   How true or false is each of the following statements for you?:
I seem to get sick a little easier than other people
I am as healthy as anybody I know
I expect my health to get worse
My health is excellent
   Do you suffer from any of the following major impairment or health problem that limits your activities?:
Arthritis or rheumatism
Back or neck problem
Cancer
Depression, anxiety or any emotional problem
Vision problem
Fractures, bone/joint injury
Hearing problem
Breathing problem
Walking problem
Other impairment or problem
   During the past 30 days, for about how many days:
was your physical health not good?
did pain make it hard for you to do your usual activities, such as self-care, work, or recreation?
have you felt sad, blue or depressed?
have you felt worried, tense or anxious?
have you felt you did not get enough rest or sleep?
have you felt very healthy and full of energy?
have you been at very nervous person?
have you felt so down in the dumps that nothing could cheer you up?
have you felt calm and peaceful?
did you have a lot of energy?
have you felt downhearted and blue?
did you feel worn out?
have you been a happy person?
did you feel tired?
   How satisfied are you with:
your sleep?
your ability to perform your daily living activities?
your capacity for work?
yourself?
your personal relationships?
your sex life?
the support you get from your friends?
the conditions of your living place?
your access to health services?
your transport?
   Are you limited in any of the following activities because of your health?:
Vigorous activities, such as running, lifting heavy objects, participating in strenuous sports
Moderate activities, such as moving a table, pushing a vacuum cleaner, bowling, or playing golf
Lifting or carrying groceries
Climbing several flights of stairs
Climbing one flight of stairs
Bending, kneeling or stooping
Walking more than a mile
Walking several blocks
Waking one block
Bathing or dressing yourself

This list above is just a sample of questions that can be presented to a user. The user's responses to the questions are assigned a value. For example, each of the multiple choice responses can be assigned a particular value, and all of the user's response can be tallied to generate a score. In addition, different questions and different responses can be weighted differently since some questions, or the severity of the response, can have a greater predictor of the user's health. The system can also assign a value based on the user's response to a combination of questions, because certain combinations can be more predictive of health. Accordingly, by assessing the user's responses to the questionnaire a Quality of Life Model score can be derived. Preferably, the Quality of Life Model score is a numerical value in the range of 0 to 1000.

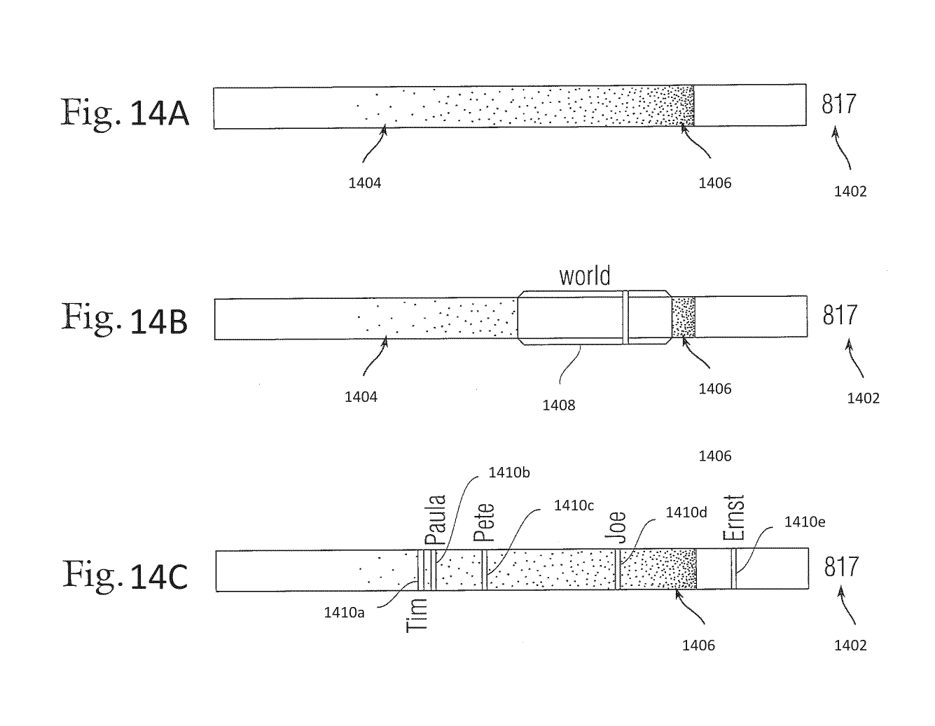

The health score is computed as a weighted average of the Metric Heath Model score and the Quality of Life Model score. The health score can be presented to the user. The health score can be presented as a numerical value, as a graphic value (i.e. as a meter, bar, or slider), or a combination of the both, for example. Referring to FIG. 14A, the health score is presented by a combination of a numerical score 1402 and a slider 1404. The slider can also be color-coded to indicate the score. The position of the slider bar 1406 indicates the user's score.

One advantage of the presentation of the health score is that it is not necessary to present the survival probabilities and raw metrics to the user. instead, users ate presented with a standardized score Preferably, this is true of the overall Metric Heath Model and Quality of Life scores, but it is also true of the relevant model inputs. This is done mainly to standardize all output, in the sense that users do not need to know whether high values of a particular input variable are good or bad; in all cases, high scores of any input value lead to higher overall health score values, and low input variable scores lead to lower overall values of the health score.

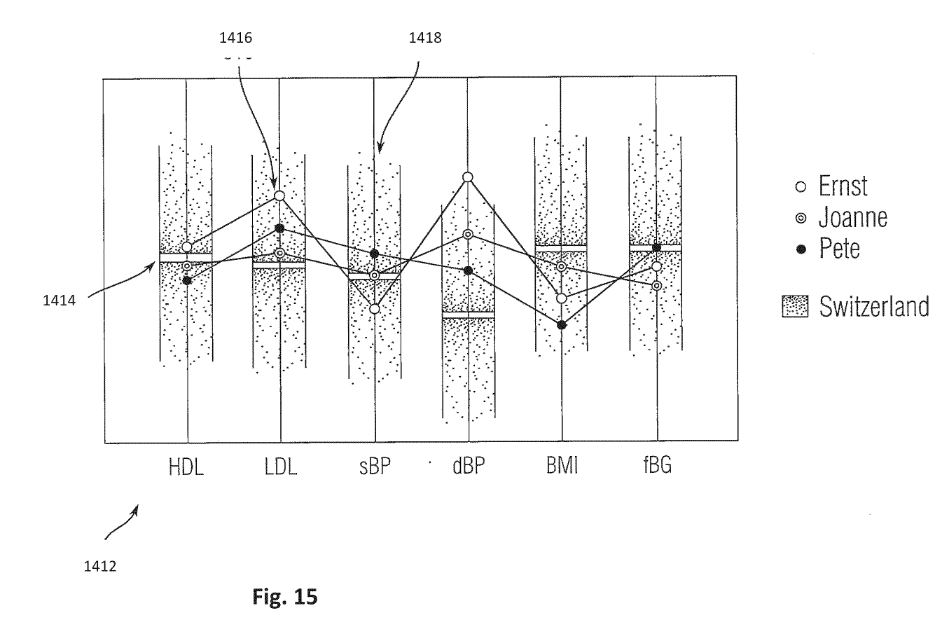

Furthermore, another advantage of the standardized health scores is that users can compare health scores against other users. This allows for comparative bench marking (against friends, co-workers, etc.) with other users. Such score comparisons can be a part of a game component of the system in which the user competes against other users, as will be described in more detail below. Gaming aspects of the system can be used motivate the user of the health score system, such as a comparison of scores among user-selected groups, comparison of individual scores within configurable subpopulation distributions, time-tracking of scores, and setting of goals, among others. Referring to FIG. 14B, the users numerical score 1402 and graphical score 1406 are presented in combination with a range of scores 1408 from a group (e.g. the world) so that the user can see how his/her score compares to others in the group. The gaming incentives can be extended by users to allow the comparison of health scores between users that can differ substantially in one or more of several specific input parameters, such as age, weight, and prior risk conditions. The system highlights improvements in modifiable user metrics, particularly in lifestyle components, and these improvements in score provide user incentives. This allows fair competition between users of a father and his children, for example, via the health score. In one aspect, the health score provides equalization between users of different characteristics and is thus similar to that of a handicap in some sports. Referring to FIG. 14C, the user's score 1406 is compared to the scores 1410a-e of a user selected group of friends. Referring to FIG. 15, the user's individual medical parameters (e,g,, the medical data provided as a part the Metric Health Model) can be compared against other users graphically without revealing the underlying actual values. The high-density lipoprotein (HDL) level, low-density lipoprotein (LDL) level, systolic blood pressure (sBP), diastolic blood pressure (dBP), body mass index (BMI), and fasting blood glucose (fBG) level are shown on a graph 1412. FIG. 15 illustrates the user's scores are represented by a line 1414, the user's friends scores are each represented by a different dot 1416, and a distribution block 1418 for a larger population group (e.g., Switzerland) is also shown. Thus, the user can compare their individual parameters to a group of friends and the average for a large population group.

Users can input data into the system at the time of an event (i.e., exercise event, food consumption, blood pressure measurement, etc.), and see the resulting update of their health score in real-time. The system can include drill-down capabilities, allowing users to see the various health score component scores, including tracking over time and the corresponding trends in all scores; it also includes the setting of goals on the various scores.

As an example of use of the system, upon registration with the system (e.g., the initial use of the system), a user is prompted to provide medical history data. The user is also prompted to respond to a complete Quality of Life questionnaire selected by the system for the given user based on the medical history and user parameters supplied by the user. After the registration, at periodic intervals, users are presented with short subsets (3 to 5 questions) of their custom Quality of Life questionnaire to keep their responses up to date and track changes. Users can enter inputs for Metric Health Model at any time, and the system prompts the user for values that have not been updated for some time. Inputs to the Metric Health Model can be acquired automatically by the system by accessing a series of digital measuring devices that have been integrated into the system (e.g., the system can comprise a mobile electronic communication device, for example, a smart phone that is in wireless communication with a measurement device, such as a blood glucose monitor, so that parameters can be measured, transmitted, and stored by the system). These can include weight, blood glucose, physical activity, and other parameters. Several or multifunction digital measurement devices can be included in the system. in the ease of medical parameters that are more difficult to obtain with a home measuring device, such as serum lipid concentration levels, users are only prompted to provide the relevant data once per (system) configured time period (e.g., annually and coinciding with a user's routine physical medical examination).

To avoid false scores, the system can include several algorithms to assess the validity of user inputs. The validation methods can range from ones based on outlier detection. to ones based on multidimensional likelihood estimators. When the system detects a possible had input value it flags it and prompts the user to either confirm. the value or to enter a new one.

The system can generate all its scores, even when missing one or more inputs. It does this by imputing the missing value or values using a variety of statistical methods that range from ones based on global population statistics, to methods based on the use of more complicated statistical models that are built into the platform. However, whenever inputs include imputed values, the system clearly flags all affected scores, and periodically alerts the user to provide the missing data. The system can also allow for score simulation, in which the user can temporarily adjust his or her parameters so that a user can see how changing certain parameters e.g., losing weight) affects the user's score.

The system can also provide recommendations to the users to take certain actions that can improve the user's health score. These recommendations can be very specific when any input variable is in its danger zone, and more generic when any input variable is outside its optimal range.

As discussed above, the health score can be used as a part of a game or competition aspect of the system. The game aspect increases the fun element of the system for the user and increases the user's affinity to continue to use the system. The game aspect can come in the form of obtaining higher levels based on achievements, competing against others (e.g., in a league), and/or convicting challenges, The “level” is an overall indication of progress. The level can be monotonically increasing and will increase by gaining activity points. Activity points can be gained from performing numerous activities, such as time spent performing fitness activities (e.g., exercising), improving one's health score, improving one's BMI, taking part in discussions on the system (e.g., the system can be a web-based social networking platform and discussions or “classes” can be offered to teach fitness skills). A user's level can be displayed on a user's profile and in discussion posts so that other users can see each other's level. A user's level status can also provide access to specific items, system features and functionality, or rewards (e.g., branded apparel).

Users can also compete within leagues in the system. The leagues are composed of groups of users and the users within the league can compete against each other (as part of a team or individually). The leagues can compete for a limited time (e.g., monthly) and the leagues can be designated based on the level of the users (using the level of the user as discussed above), the type of activity being performed in the league, and the geographic region of the users. For example, one particular league can be the “bronze” (level) “mountain biking” (sport) “Greater Zurich Area” (region) league and a user's success in this league is measured by the distance traveled and elevation climbed (measured quantity). Thus, bronze level users living in the Greater Zurich Area that are interested in mountain biking can compete in this league. Limiting the leagues to a. particular region gives the users something to relate with and all the users can share in common, and further allows users to meet face to face (e.g., for group exercise events), One issue with one big international league is that such a league may seem anonymous, crowded and meaningless to some users (members competing against members residing on completely different continents with language barriers can inhibit group or team mentalities). Limiting leagues to particular level brackets equalizes the playing, field for users of particular skill Quantities to be measured to determine performance in the league can include distance (horizontal, vertical) and duration of fitness activity performed, for example. Users can also form teams within the leagues. Team leagues work in the same way as the leagues outlined above, however the ranking is based on the team's overall performance. Teams increase the communal aspect of participation in the activity. Teams can be fixed in size (e.g., 2, 3, 5, 10, etc. users).

Users can also be presented by the system with challenges to complete. The challenges can set forth a time period for completion of a goal. The goals of the challenge can be, for example, health score improvement (normalized), completion of sport-related activity parameters (e.g., total distance, total climbing, etc.), or completion of a sport-related activity within a specific period of time (e.g., complete six minute mile on a specific route). The challenge can be public and any user can participate, or limited to a group (e.g., friends, co-workers, social group, etc.) As an example, a particular public challenge can be an inline skating challenge in New York City for the route around the Central Park Loop measuring the time taken for completion. Public challenges can be generated automatically by the system or by system administrators. Group challenges can be issued by group members. Challenges provide strong appointment dynamics, encouraging users to commit to exercise. Challenges (typically) have a lower time commitment than leagues. Route selection cart be automated with the community. in a first step, the community can publish routes on the system platform (e.g., a social networking type website); in a second step, the system selects popular routes (i.e. routes with high user activity) as weekly challenges. Route validation is done by GPS tracking. Challenges can be safety screened to prevent the promotion of unduly risky challenge activities, such mountain biking dangerous downhill routes.

The league and challenge systems provide opportunities to grant achievements. Achievement status indications can be collected and displayed on a user's profile. Achievements are much like a trophy, medal, or award provided to the user for completing challenges and/or succeeding in a league activity. Many different achievements are possible, such as related to the number of friends the user has on the system (community participation), a achievements related to the time, intensity, and number of fitness activities engaged in (level of fitness participation), achievements related to specific sport activities (e.g., distance run), the frequency a user measures their parameters (e.g., weight) in order to keep the system up to date, the amount of weight lost, or the ability to maintain ones BMI, for example. The following list is an exemplary set of achievements and the activities required to earn the achievements:

### Exemplary Achievement List:

Challenger: Take part in a public challenge.
Accomplished Challenger: Take part in 10 public challenges.
Champion: Win a challenge.
Multi-sport Champion: Win a public challenge in two different sports.
International Challenger: Take part in a public challenge in two different countries.
International Champion: Win a public challenge in two different countries.
World Wide Challenger: Take part in a public challenge on each continent.
World Wide Champion: Win a public challenge on each continent.

Other aspects of the challenge and league systems are that the systems can be tied to marketing opportunities. For example, marketers can sponsor prizes for the winners of a challenge. The prize can be related to the challenge (e.g., gift certificate to health food score for winner of weight loss challenge). In addition, challenge routes can be selected to direct users to certain areas to increase tourism or to begin/end at selected destinations (e.g., bike challenge begins in front of sports equipment store).

One advantage of the system is that it provides users and groups of users with benchmarking capabilities. It allows other groups, such as insurance carriers or employers, to assess the relative health of individuals in order to determine the health related risks of each individual. Accordingly users can compare themselves against others in order to assess their comparative health level amongst a group of friends. Insurance carriers can use the health score information to set premiums for an individual or a group of individuals (e.g. employees of a company). In other implementations, health scores can be provided for a group based on the health scores of the individuals in the group. For example, a health score can be calculated for a company based on its employees so that an insurance carrier can set premiums based on the health score of the company compared to other companies. In further applications, the health score can be used for assessing the health of professional athletes to determine the athlete's real market value. Vast amounts of money and resources are invested in athletes at all levels in professional sports. A large component of the decision about investing in an athlete is based on the past performance of the athlete. Other factors can include past physical injury history and the athlete submitting, to a physical exam before the deal is complete. The health score can be used as an indicator of the athlete's current health and used as a predictor of the athlete's future performance. If the athlete's health score were low, this can indicate that the athlete is more prone to suffering an injury or that physical performance will diminish. Accordingly, the health score can form a basis for a decision on whether to invest in an athlete. The health scores could also be used as a predictor of the outcome of a particular game played between two teams, For example, the health scores of the individual team members can be aggregated. in order to provide a team health score. A comparison of the team health scores can be indicative of the likely outcome of the game between the two teams (e.g., the team with highest health score may be more likely to win). Such information can be used in gaming contexts such as fantasy sports teams, or in order to set odds for sports betting. The health score could be used for club competitions (e.g., group health improvement competitions, advertising based on a person's health score, gaming, TV/internet, etc.

Thus, in a broad aspect, a method according to the invention can be understood as collecting health related information, processing the information into a health score, and publishing the health score is provided. A system for implementing the method can include a computer having, a processor, memory, and code modules executing in the processor for the collection, processing, and publishing of the information, Information concerning a plurality of health related parameters of a user is collected, particularly, both intrinsic values concerning the measurable, medical parameters of at least one natural person, and the extrinsic values concerning the activities of each such person(s) such as the exercise performed, the type of job the person has and the amount of physical work associated with the job (e.g. sedentary, desk job versus active, manual labor intensive job) and/or the calories/food consumed. Weighting factors are applied to the health related parameter in order control the relative affect each parameter has on the user's calculated health score. The health score is computed using the processor by combining the weighted parameters in accordance with an algorithm. The health score is published to a designated group via a portal. In one implementation, the portal is an internet based information sharing forum.

Thus, although present invention has been described in relation to particular embodiments thereof, many other variations and modifications and other uses will become apparent to those skilled in the art. It is preferred, therefore, that the present invention not b limited by the specific disclosure herein.

What is claimed:

1. A wearable device that is configured to provide user information, and to transmit the user information to a remote computing device, and to receive from the remote computing device health-related information associated with the user information for outputting to a user, the wearable device comprising:
a display configured to provide a user interface;
at least one sensor that is respectively configured to sense information while the wearable device is being worn by the user, wherein the sensed information is of the type that is associated with biological features, physiological features and/or physical activity of the user;
a communications subsystem that is configured to communicate with the remote computing device; and
a processing subsystem including a processor and processor readable media, the processing subsystem configured to:
process the sensed information from the at least one sensor to provide processed user information;
transmit via the communication subsystem, to the remote computing device, the processed user information;
receive, from the remote computing device, the health-related information associated with the processed user information; and
provide, via the user interface on the display, the health-related information received from the remote computing device.

2. The wearable device of claim 1,
wherein the health-related information is formatted as a health score that is calculated by applying a decay component to reduce a relative weight of the sensed information associated with the physical activity of the user and further transforming the processed user data into a masked composite numerical value; and
further wherein the health score is provided via the user interface on the display free of human intervention.

3. The wearable device of claim 1, wherein the processing subsystem is further configured to:
process second sensed information from the at least one sensor to provide processed second user information;
transmit via the communication subsystem, to the remote computing device, the processed second user information;
receive, from the remote computing device, second health-related information associated with the processed user information; and
provide, via the user interface on the display, the second health-related information received from the remote computing device.

4. The wearable device of claim 3, wherein the processing subsystem is further configured to track health-related information over time.

5. The wearable device of claim 1, wherein the physical activity of the user includes one or more of walking, running, swimming and sleeping.

6. The wearable device of claim 1, wherein the physiological features includes one or more of temperature, blood pressure, blood glucose level, perspiration, and heart rate.

7. The wearable device of claim 1, wherein the user interface is configured to receive information input by the user.

8. The wearable device of claim 7, wherein the information is received by scanning a bar code.

9. The wearable device of claim 7, wherein the information input by the user is associated with food, and further wherein the processing subsystem is further configured to process the information input by the user to calculate caloric expenditure.

10. The wearable device of claim 1, wherein the at least one sensor includes a pedometer, a galvanic skin sensor a biosensor, a GPS unit, an altimeter, a cadence sensor, a velocity sensor and/or a heart rate monitor.

11. The wearable device of claim 1, wherein the processing system is further configured to display a rank of health scores respectively associated with a plurality of users.

12. The wearable device of claim 1, wherein the communication subsystem is configured to communicate with the remote computing device via at least one of BLUETOOTH, Wi-Fi, Near Field Communications, ANT and ZigBee.

13. The wearable device of claim 1, further comprising an accelerometer, and wherein orientation of information displayed on the display is rotated or inverted as a function of the accelerometer.

14. The wearable device of claim 13, wherein the accelerometer is a three-axis accelerometer.

15. The wearable device of claim 1, wherein the display includes backlighting.

16. The wearable device of claim 1, wherein the device is configured as a strap.

17. The wearable device of claim 16, wherein the strap is adjustable.

18. The wearable device of claim 1, wherein the processing subsystem is further configured to display the processed user information.

19. A method for providing user information via a wearable device that is configured to transmit the user information to a remote computing device, and to receive from the remote computing device health-related information associated with the user information for outputting to a user, the method comprising:
providing a user interface via at least a display configured with the wearable device;
sensing, by at least one sensor configured with the wearable device, information while the wearable device is being worn by the user, wherein the sensed information is of the type that is associated with biological features, physiological features and/or physical activity of the user;
communicating, via a communication system, with the remote computing device;
processing via a processing subsystem that includes a processor and processor readable media, the sensed information from the at least one sensor to provide processed user information;
transmitting via the communication subsystem, to the remote computing device, the processed user information;
receiving, from the remote computing device, the health-related information associated with the processed user information; and
providing, via the user interface on the display, the health-related information received from the remote computing device.

20. The method of claim 19,
wherein the health-related information is formatted as a health score that is calculated by applying a decay component to reduce a relative weight of the sensed information associated with the physical activity of the user and further transforming the processed user data into a masked composite numerical value; and
providing, via the user interface on the display, the health score free of human intervention.
